(19) United States
(12) Patent Application Publication
(10) Pub. No.: US 2020/0101313 A1
(43) Pub. Date: Apr. 2, 2020

(54) PHOTOTHERAPY DEVICE AND PHOTOTHERAPY METHOD

(71) Applicants: PUBLIC UNIVERSITY CORPORATION NAGOYA CITY UNIVERSITY, Nagoya-city (JP); Ushio Denki Kabushiki Kaisha, Tokyo (JP)

(72) Inventors: Akimichi MORITA, Aichi (JP); Hideyuki MASUDA, Tokyo (JP); Makoto KIMURA, Tokyo (JP)

(73) Assignees: PUBLIC UNIVERSITY CORPORATION NAGOYA CITY UNIVERSITY, Nagoya-city (JP); Ushio Denki Kabushiki Kaisha, Tokyo (JP)

(21) Appl. No.: 16/582,062

(22) Filed: Sep. 25, 2019

(30) Foreign Application Priority Data
Sep. 28, 2018 (JP) 2018-184050

(51) Int. Cl. A61N 5/06

(52) U.S. Cl. CPC A61N 5/0616; A61N 2005/0661; A61N 2005/0652

(57) ABSTRACT

A phototherapy device includes a light source module configured to emit therapeutic light to an affected area of a skin. The light source module includes a plurality of LED elements to emit light having a peak wavelength in a range of 365 nm±5 nm, and having a wavelength equal to or shorter than 350 nm. The light source module also includes a light outlet window to which the light emitted from the LED elements is incident and through which the therapeutic light exits. The light source module also includes a filter configured to substantially shield light having a wavelength equal to or shorter than 350 nm when the light emitted from the LED elements passes through the filter.

### 1. TECHNICAL FIELD

The present invention relates to phototherapy devices and phototherapy methods for treating skin diseases through irradiation of ultraviolet light.

### 2. DESCRIPTION OF THE RELATED ART

As a method of treating skin diseases, a technique where an affected area is irradiated with ultraviolet light (rays) is widely used in recent years. One skin-disease phototherapy device that is in practical use for treating skin diseases with ultraviolet light includes a discharge lamp serving as an ultraviolet light source and emits ultraviolet light (UVA1) having a wavelength in a range of 340 nm-400 nm (SELLAMED 2000 System Dr. Sellmeier, for example). A certain type of this skin-disease phototherapy device for treating skin diseases that is in practical use is configured to irradiate an affected area with light from a metal halide lamp via a plurality of (three, for example) wavelength selective filters, resulting in a low utilization efficiency of the light (ultraviolet light) from the ultraviolet light source.

Accordingly, proposed is the use of an LED element as an ultraviolet light source in a skin-disease phototherapy device for treating skin diseases with ultraviolet light (for example, Japanese Patent Application Laid-Open Publication No. Hei10-190058 and Japanese Patent Application Laid-Open Publication No. 2007-151807).

A skin-disease phototherapy device of Japanese Patent Application Laid-Open Publication No. Hei10-190058 includes a plurality of LED elements disposed on a circular substrate or on a concave surface of a case. Japanese Patent Application Laid-Open Publication No. 2007-151807 indicates that an LED element having a peak wavelength in a range of 350 nm-390 nm is effective in treating refractory eczema, dyshidrotic eczema, cutaneous T cell lymphoma, atopic dermatitis, alopecia areata, keloids, cicatrices, atrophic striae, and scleroderma.

Long-wavelength ultraviolet light having a wavelength of 320 nm-400 nm is known to have an immediate pigment darkening action of darkening skins immediately upon irradiation. The document by C. IRWIN, A. BARNES, D. VERES, K. KAIDBEY, “An ultraviolet light action spectrum for immediate pigment darkening,” Photochemistry and Photobiology, Vol. 57, No. 3, pp. 504-507, 1993 shows an action spectrum with respect to skins and points out that the immediate pigment darkening action peaks at a wavelength of 340 nm. With immediate pigment darkening, a skin is darkened immediately upon being irradiated with long-wavelength ultraviolet light, and the skin then returns to its original condition in several hours or in several days. However, when immediate pigment darkening is repeated continually, long-lasting pigmentation may occur.

### SUMMARY OF THE INVENTION

The present inventors have conducted diligent studies and found that, with regard to a light source to be used in a phototherapy device for scleroderma, an LED element that emits light having a peak wavelength of 365 nm yields the highest effect. Accordingly, the use of an LED element that emits light having a peak wavelength of 365 nm as a light source can bring about a superior therapeutic effect efficiently against scleroderma.

A skin disease to which an ultraviolet light therapy is applied spreads over an area of from several tens of square centimeters to several hundreds of square centimeters. Therefore, a phototherapy device where an LED light source is used requires a plurality of LED elements.

However, even when a plurality of LED elements each manufactured to attain a peak wavelength of 365 nm are used as an LED light source, the peak wavelength of the light emitted from the LED light source may vary by around ±5 nm due to the manufacturing variations (tolerance) of the LED elements. In that case, if the peak wavelength shifts to the shorter wavelength side from 365 nm, the quantity of light emitted at around 340 nm increases, and this causes an immediate pigment darkening reaction to occur more easily. For example, with regard to scleroderma, one conceivable symptomatic treatment includes repeated irradiation with long-wavelength ultraviolet light. Then, immediate pigment darkening is repeated, and long-lasting pigmentation is highly likely to occur. Such pigmentation increases the absorption of light by the epidermis and reduces the invasiveness (deep penetration) of the light into the dermis, or the target. This deteriorates the therapeutic efficiency.

One object of the present invention is to provide a phototherapy device that can bring about a superior skin therapy effect while suppressing an immediate pigment darkening action. Another object of the present invention is to provide a phototherapy method that can bring about a superior skin therapy effect while suppressing an immediate pigment darkening action.

According to one aspect of the present invention, there is provided a phototherapy device that includes a light source module configured to emit therapeutic light to an affected area of a skin. The light source module includes a plurality of LED elements to emit light having a peak wavelength in a range of 365 nm±5 nm, and having a wavelength equal to or shorter than 350 nm. The light source module also includes a light outlet window to which the light emitted from the LED elements is incident and through which the therapeutic light exits. The light source module also includes a filter configured to substantially shield light having a wavelength equal to or shorter than 350 nm when the light emitted from the LED elements passes through the filter.

As mentioned above, the light source module includes a plurality of LED elements to emit light. Light having a wavelength of 365 nm brings about the greatest therapeutic effect to scleroderma. Because the LED elements emit light having a peak wavelength of 365 nm±5 nm, the phototherapy device can provide a significant therapeutic effect to scleroderma. Unlike emission line spectrum of a discharge lamp, for example, the peak wavelengths of the LED elements may vary in a range of approximately ±5 nm due to manufacturing tolerance (variations such as a band gap). Because the phototherapy device of the invention has the filter that substantially blocks the light having a wavelength equal to or shorter than 350 nm when the light emitted from the LED elements passes through the filter, it is possible to remove light having a wavelength around 340 nm, which would otherwise trigger the immediate pigment darkening action, from the therapeutic light even if the peak wavelengths of the LED elements are at the lower limit of the acceptable wavelength. Thus, it is possible to reduce the immediate pigment darkening action. As a result, it is possible to suppress the pigmentation which would take place upon repeating of the immediate pigment darkening action. Accordingly, it is possible to maintain the deep penetration of the light to the target, i.e., dermis, and maintain the therapeutic efficiency.

The therapeutic device of the invention can, therefore, suppress the immediate pigment darkening action while providing a significant skin therapeutic effect to the scleroderma.

The filter may substantially shield light having a wavelength equal to or shorter than 355 nm when the light emitted from the LED elements passes through the filter. By substantially excluding the light having a wavelength of 355 nm and below from the light emitted from the LED elements and using the remaining light as the therapeutic light, the therapeutic effect becomes greater than the side effect (immediate pigment darkening action).

The LED elements may have different peak wavelengths, respectively, in a range of 365 nm±5 nm. Even if the LED elements have variations in the peak wavelength, such LED elements can be used as the light source elements without any adjustments and replacement. In other words, selection and replacement of LED elements or the like is not necessary in order to prepare the LED elements that have the same peak wavelength.

The filter may be a colored glass filter. If the filter is a colored glass filter, such filter can improve the efficiency of utilization of light, as compared to a case where the filter is a dielectric multi-layer filter. Because the dielectric multi-layer filter has the incident angle dependency, it is necessary to improve the angle characteristics of the light emitted from the LED elements with a collimator lens, if the efficiency of utilization of light should be improved and the dielectric multi-layer filter should be used. This makes the device configuration complicated, and makes the device size large. In contrast, if the colored glass filter is used as the filter, the device configuration becomes simple and the device size becomes small.

The colored glass filter may have a 5% transmittance at a first wavelength and a 72% transmittance at a second wavelength, and a midpoint between the first wavelength and the second wavelength may be between 350 nm, inclusive, and 365 nm, inclusive. A difference between the first wavelength and the second wavelength may be equal to or shorter than 30 nm. When such configuration is employed, it is possible to cut the wavelength (unnecessary wavelength) that would increase the risk of triggering the immediate pigment darkening action while transmitting the light having a wavelength (effective wavelength) that enhances the therapeutic effect.

The first wavelength may be equal to or shorter than 340 nm. Such filter can properly transmit the light having the effective wavelength.

The colored glass filter may have an average transmittance of 80% or more in a wavelength range between the second wavelength, inclusive, and 800 nm, inclusive. Such filter can efficiently extract the light having the effective wavelength.

An irradiance of the therapeutic light immediately below (downstream of) the light outlet window may be between 33 mW/cm², inclusive, and 150 mW/cm², inclusive. When the irradiance of the therapeutic light immediately below the light outlet window is equal to or greater than 33 mW/cm², it is possible to reduce the treatment time. When the irradiance of the therapeutic light immediately below the light outlet window is equal to or smaller than 150 mW/cm², it is possible to reduce the heat and heat-derived pain, which a patient feels during the treatment.

According to another aspect of the present invention, there is provided a method of emitting therapeutic light to an affected area of a skin from a light source module having a plurality of LED elements. The method includes causing the LED elements of the light source module to emit light having a peak wavelength in a range of 365 nm±5 nm, and having a wavelength equal to or shorter than 350 nm. The method also includes substantially shielding, by a filter, light having a wavelength equal to or shorter than 350 nm when the light emitted from the LED elements passes through the filter. The method also includes directing the light, which has been emitted from the LED elements and has passed through the filter, to the affected area as the therapeutic light.

This method can suppress the immediate pigment darkening action while providing a remarkable skin therapeutic effect to the scleroderma.

### BRIEF DESCRIPTION OF THE DRAWINGS

FIG. 1 illustrates an overall configuration of an exemplary phototherapy device according to an embodiment of the present invention.

FIG. 2 illustrates a configuration of a light source module of the phototherapy device.

FIG. 3A is a view useful to describe a mechanism of immediate pigment darkening action, and particularly shows a structure of a human skin.

FIG. 3B is another view useful to describe the mechanism of immediate pigment darkening action, and particularly shows a melanin that is photo-oxidized and darkened upon UV irradiation.

FIG. 3C is still another view useful to describe the mechanism of immediate pigment darkening action, and particularly shows that a dark melanin is reduced and returns to its original state.

FIG. 4A to FIG. 4F is a series of views useful to describe how a pigmentation takes place as UV irradiation is repeated.

FIG. 5 shows action spectrum of immediate pigment darkening, filter characteristics and the light source module.

FIG. 6 is a view useful to describe transmission characteristics of a filter.

FIG. 7A shows spectra obtained after light passes through filters.

FIG. 7B is an enlarged view of a part of FIG. 7A.

FIG. 8 shows a result of Experiment 1.

FIG. 9 shows a result of Experiment 2.

FIG. 10A shows light absorption when no pigmentation occurs.

FIG. 10B shows light absorption when pigmentation has occurred.

FIG. 11 shows a modification to the light source module.

### DETAILED DESCRIPTION OF EMBODIMENTS

An embodiment of the present invention will be described with reference to the drawings.

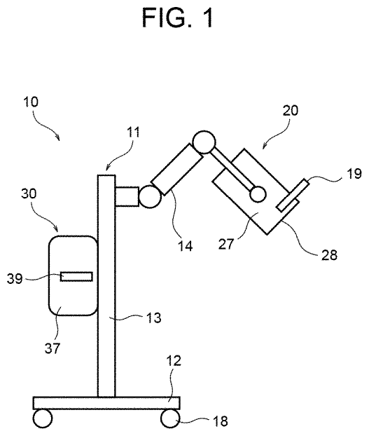

FIG. 1 shows a general configuration of an exemplary phototherapy device 10 according to the embodiment.

The phototherapy device 10 is a skin-disease therapy device that performs a UVA1 therapy of irradiating an affected area with therapeutic light having a spectrum in a UVA1 range.

In the UVA1 therapy, ultraviolet light of 340 nm-400 nm is used, and a characteristic aspect of the UVA1 therapy lies in that the ultraviolet light reaches deep under the skin, as compared to a therapeutic technique where ultraviolet light in a UV-B range is used. Therefore, the UVA1 therapy is effective against a disease having its cause in the dermis and is known to be highly effective against atopy, prurigo, and scleroderma.

In this embodiment, the phototherapy device 10 is a therapy device for treating scleroderma.

The phototherapy device 10 includes a light source module (light source block) 20 and a control module (control block) 30 that controls the light source module 20. The light source module 20 and the control module 30 are supported by a support 11.

The support 11 includes a mount 12, a support column 13, and an operable arm 14. The mount 12 is supported by wheels 18 on a floor surface. The support column 13 extends upward from a center portion of the mount 12. The operable arm 14, allowing the light source module 20 to freely reciprocate relative to the support column 13, supports the light source module 20 at an upper end portion of the support column 13. In the support 11, the light source module 20 is attached to a leading end portion of the operable arm 14. The control module 30 is attached to a middle portion of the support column 13 by a fixing member (not illustrated).

The light source module 20 includes, for example, a rectangular parallelepiped case 27 and a window member 28 provided in an end surface of the case 27. The light source module 20 emits therapeutic light through the window member 28. The window member 28 is a light-irradiation window (light outlet window) from which the therapeutic light exits to irradiate an affected area of a patient with the therapeutic light. The light source module 20 may be provided with a hand lever 19 for allowing an operator (e.g., a doctor) of the phototherapy device 10 to manually swing or move the light source module 20.

The control module 30 includes, for example, a rectangular parallelepiped case 37 and a graphic operation panel 39 provided on a side surface of the case 37. The graphic operation panel 39 can be operated by the operator of the phototherapy device 10.

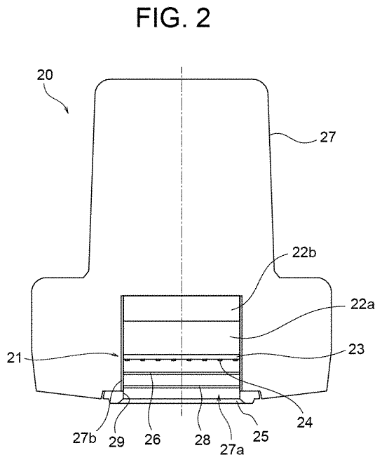

FIG. 2 illustrates a configuration of the light source module 20.

As illustrated in FIG. 2, the light source module 20 includes a light source unit 21 disposed in a housing portion 27a formed inside the case 27. The light source unit 21 is supported by a support member (not illustrated) in the housing portion 27a and is disposed to face the light-irradiation window 28. The light-irradiation window 28 is fitted in an opening 29 of the housing portion 27a.

The light source unit 21 includes a plurality of LED elements 24 serving as a light source. The LED elements 24 are disposed, for example, on a rectangular plate-like substrate 23. For example, the LED elements 24 can be disposed on the substrate 23 in a lattice pattern where the LED elements 24 are arrayed in orthogonal directions at a predetermined interval. For example, when sixty-four LED elements 24 are used, the LED elements 24 can be disposed in a lattice pattern of eight by eight in respective orthogonal directions. The number and the arrangement of the LED elements 24 are not limited to the above.

The spacing between the substrate 23 and the light-irradiation window 28 may be, for example, 40 mm.

The light source module 20 may include a heat sink 22a and an axial-flow fan 22b provided on the opposite side from the light-irradiation window 28 of the light source unit 21 in the housing portion 27a for cooling the light source unit 21.

The light source module 20 may include an irradiation attachment 25 provided to the outer side of the light-irradiation window 28 for closing the opening 29 of the housing portion 27a. The irradiation attachment 25 can prevent an affected area from making direct contact with the light-irradiation window 28. For example, the irradiation attachment 25 can have a thickness of 10 mm, and the light-irradiation window 28 can be disposed at a position 5 mm away from the irradiation attachment 25 in the housing portion 27a.

The light source module 20 may include a reflective member 27b provided on an inner surface of the housing portion 27a for reflecting light emitted from the LED elements 24. Alternatively, the inner surface of the housing portion 27a may be subjected to mirror finishing or the like and have a reflective property.

The LED elements 24 each have a peak wavelength in a wavelength range of 365 nm±5 nm (also referred to below as a “specific wavelength range”) and emit ultraviolet light including light having a wavelength of no longer than 350 nm. Specifically, the LED elements 24 emit ultraviolet light (UVA1) having a peak wavelength in the specific wavelength range and having a wavelength in a range of 340 nm-400 nm. The half width (full width at half maximum) of the optical spectrum of the LED elements 24 can be 5 nm-20 nm.

The ultraviolet light having a peak wavelength of 365 nm is most effective as therapeutic light against scleroderma. The above-mentioned specific wavelength range is a wavelength range that takes into consideration a variation in the peak wavelength of an LED element manufactured to attain a peak wavelength of 365 nm.

Unlike the emission-line spectrum of a discharge lamp, for example, the peak wavelength of an LED element may vary by around ±5 nm due to the manufacturing variations (variations such as a band gap). The specific wavelength range is set to 365 nm±5 nm in consideration of that, when a plurality of LED elements each manufactured to attain a peak wavelength of 365 nm are used as a light source, the peak wavelength of each LED element may vary within a wavelength range of 365 nm±5 nm.

For the LED elements 24, for example, surface-mounted LED elements of AlInGaN-based semiconductor can be used.

The light source unit 21 is electrically coupled to a cable (not illustrated) for supplying power to the LED elements 24 included in the light source unit 21. This cable electrically couples the light source module 20 (light source unit 21) to the control module 30.

The light source module 20 includes a filter 26 disposed between the LED elements 24 and the light-irradiation (light-outlet) window 28 in the housing portion 27a. The filter 26 substantially cuts (blocks), of the light emitted from the LED elements 24, light having a wavelength of no longer than 350 nm or preferably light having a wavelength of no longer than 355 nm. The LED elements 24 emit light in the UVA1 wavelength range (340 nm-400 nm). Therefore, the filter 26 may substantially cut light having a wavelength of no shorter than 340 nm nor longer than 350 nm or preferably light having a wavelength of no shorter than 340 nm nor longer than 355 nm.

As described above, scleroderma is most effectively treated with irradiation with ultraviolet light having a peak wavelength of 365 nm. In other words, an action spectrum for treating scleroderma peaks at a wavelength of 365 nm, and the therapeutic effect decreases as the wavelength shifts from 365 nm. Long-wavelength ultraviolet light having a wavelength of 320 nm-400 nm is known to have an immediate pigment darkening action of darkening skins immediately upon irradiation.

Now, a mechanism of immediate pigment darkening will be described.

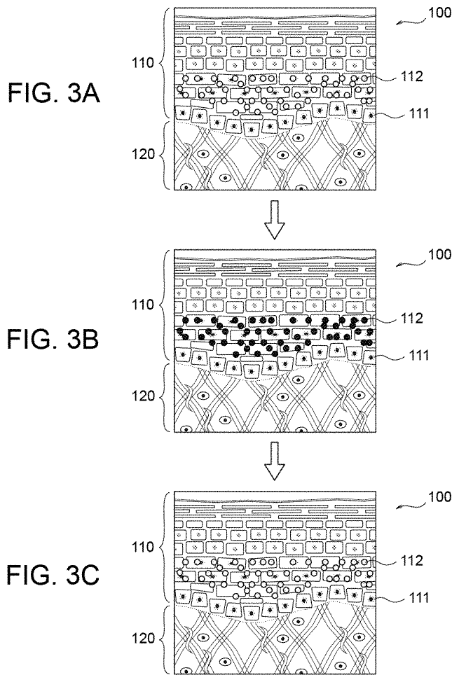

FIG. 3A shows the structure of a skin 100 of a human. As shown in FIG. 3A, a layer closest to the surface is epidermis 110, dermis 120 lies underneath the epidermis 110, and a melanocyte (pigment cell) in a basal layer 111 included in the epidermis 110 generates a melanin 112. Upon being irradiated with ultraviolet light, an existing melanin 112 is photo-oxidized and darkened, as illustrated in FIG. 3B. Thereafter, upon a prescribed time (several hours or several days) having passed, the melanin that has been darkened through photo-oxidation is reduced and returns to its original state, as illustrated in FIG. 3C. This is the mechanism of immediate pigment darkening.

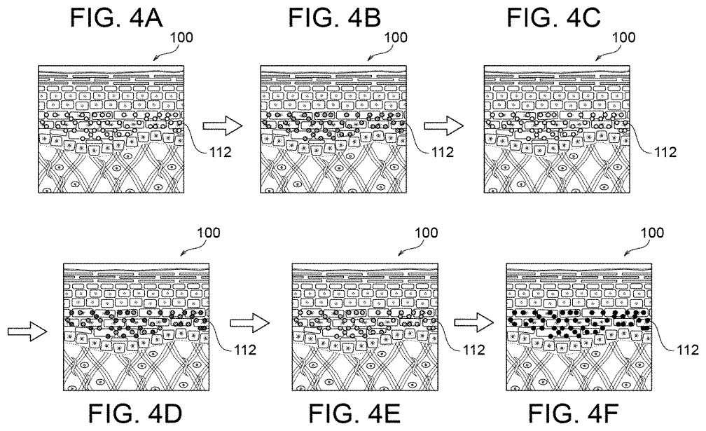

When irradiation with ultraviolet light is repeated and immediate pigment darkening is repeated, darkening of the melanin 112 builds up, as illustrated in FIGS. 4A to 4F, and pigmentation occurs. FIG. 4A changes to FIG. 4B upon UV irradiation. FIG. 4B changes to FIG. 4C as the prescribed time passes. FIG. 4C changes to FIG. 4D upon UV irradiation. FIG. 4D changes to FIG. 4E as the prescribed time passes. FIG. 4E changes to FIG. 4F upon UV irradiation.

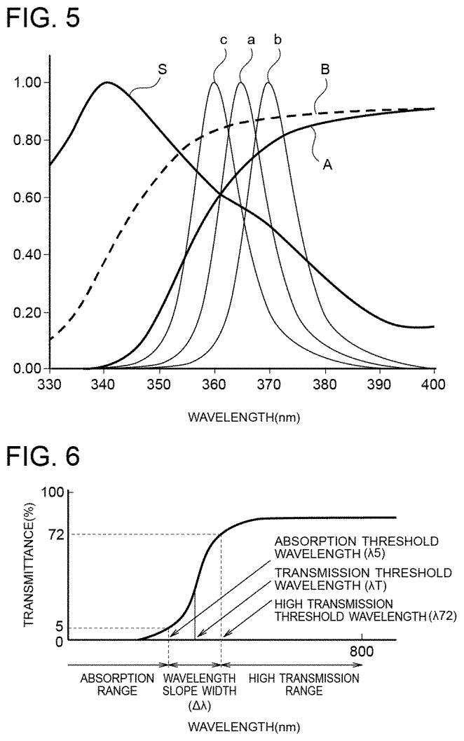

The action spectrum of immediate pigment darkening peaks at a wavelength of 340 nm, as indicated by a curve S in FIG. 5, and irradiation with ultraviolet light having a wavelength of around 340 nm is most likely to cause immediate pigment darkening.

In this embodiment, the LED elements 24 are used as the light source in the phototherapy device 10. The optical spectrum of the light emitted from the LED elements 24 is broad, as indicated by curves a to c in FIG. 5, and includes ultraviolet light having a wavelength of around 340 nm. In other words, this poses the risk of immediate pigment darkening. In FIG. 5, the curve a is the optical spectrum of an LED element having a peak wavelength of 365 nm, the curve b is the optical spectrum of an LED element having a peak wavelength of 370 nm (wavelength of 365 nm±5 nm), and the curve c is the optical spectrum of an LED element having a peak wavelength of 360 nm (wavelength of 365 nm-5 nm).

As described above, the peak wavelengths of the LED elements 24 vary by around ±5 nm. When the peak wavelength of an LED element 24 is at the lower limit of this variation (curve c), this peak wavelength is close to 340 nm, or the peak of the action spectrum of immediate pigment darkening, and thus the risk of immediate pigment darkening increases.

In order to obtain a therapeutic effect against scleroderma while reducing the risk of immediate pigment darkening, of the light emitted from the LED elements 24, light having a wavelength of around 340 nm, where the risk of immediate pigment darkening peaks, needs to be blocked while keeping light having a wavelength of around 365 nm, where the therapeutic effect peaks, from being blocked.

In this embodiment, in consideration of the balance between the therapeutic effect and the risk of immediate pigment darkening, of the light emitted from the LED elements 24, light having a wavelength of no longer than 350 nm is substantially cut with the filter 26. In other words, light in a wavelength range where the risk of immediate pigment darkening surpasses (overwhelms) the therapeutic effect is cut. In addition, of the light emitted from the LED elements 24, light having a wavelength of no longer than 355 nm is substantially cut, and this can ensure that the therapeutic effect surpasses the risk of immediate pigment darkening.

The filter 26 may be a colored glass filter. If the filter 26 is a colored glass filter, materials that can be used for the filter 26 include silicate glass and phosphate glass.

FIG. 6 is a view useful to describe parameters relevant to transmission characteristics of a colored glass filter.

The filter 26 can have filter characteristics where a transmission threshold wavelength (λT) is no shorter than 350 nm nor longer than 365 nm and a wavelength slope width (Δλ) is no greater than 30 nm. The filter 26 can also have filter characteristics where a high-transmission-range transmittance (TH) is no lower than 80%. Furthermore, the filter 26 can have filter characteristics where an absorption threshold wavelength (λ5) is no longer than 340 nm.

As illustrated in FIG. 6, the transmission threshold wavelength (λT) is a wavelength at a midpoint between a first wavelength where the transmittance is 5% (absorption threshold wavelength (λ5)) and a second wavelength where the transmittance is 72% (high-transmission threshold wavelength (λ72)). The wavelength slope width (Δλ) is the distance between the first wavelength (absorption threshold wavelength (λ5)) and the second wavelength (high-transmission threshold wavelength (λ72)). The high-transmission-range transmittance (TH) is a mean transmittance within a high-transmission range (a wavelength range from the high-transmission threshold wavelength (λ72) to 800 nm).

When the transmission threshold wavelength (λT) is short, the wavelength where the risk of immediate pigment darkening increases (unwanted wavelength) cannot be cut. When the transmission threshold wavelength (λT) is long, the wavelength where the therapeutic effect increases (effective wavelength) is cut. Accordingly, λT is preferably no shorter than 350 nm nor longer than 365 nm. When the wavelength slope width (Δλ) is too large, the effective wavelength and the unwanted wavelength cannot be separated, and thus Δλ is preferably no greater than 30 nm. When the high-transmission-range transmittance (TH) is too low, the effective wavelength cannot be extracted efficiently, and thus TH is preferably no lower than 80%. When the absorption threshold wavelength (λ5) is long, the effective wavelength is cut, and thus λ5 is preferably no longer than 340 nm.

In this manner, the light source module 20 includes the LED elements 24 that emit light having a peak wavelength in a wavelength range of 365 nm±5 nm, and the filter 26 receives the light emitted from the LED elements 24, substantially cuts, of the received light, light having a wavelength of no longer than 350 nm or preferably light having a wavelength of no longer than 355 nm, and emits light transmitted therethrough as therapeutic light. The therapeutic light transmitted through the filter 26 is emitted through the light-irradiation window 28.

The light-irradiation window 28 preferably has a light-transmitting property with respect to the therapeutic light and has a high mechanical strength. Specific examples of the material for the light-irradiation window 28 include silica glass. When the light-irradiation window 28 is made of silica glass, the light-irradiation window 28 can be provided with a high mechanical strength, and possible damage that could be caused by an impact can be prevented. In addition, even if the light-irradiation window 28 is stained, the light-irradiation window 28 can be cleaned easily with alcohol or the like.

In the control module 30, an LED-driving power source unit and a control unit such as a programmable logic controller (PLC) are disposed inside the case 37. The control module 30 can control the driving of the LED elements 24 included in the light source module 20 and control the irradiance, the emissive duration, and so on of the light emitted from the light source module 20.

The irradiance obtained directly underneath the light-irradiation window 28 is preferably no lower than 33 mW/cm² nor higher than 150 mW/cm² (i.e., between 33 mW/cm² inclusive and 150 mW/cm² inclusive). A range considered to be directly underneath the light-irradiation window 28 is a range of less than 3 cm from the light-irradiation window 28, for example.

The irradiance of 33 mW/cm² is an irradiance required to obtain an ultraviolet-light irradiation amount (cumulative irradiation amount or dose) of 60 J/cm² that is required to treat scleroderma in a therapy duration (irradiation duration) of 30 minutes. Typically, a therapy device for skin diseases is required to finish a therapy within 30 minutes. When the irradiance is raised too much, the patient may experience heat and/or pain caused by heat during a therapy. The irradiance of 150 mW/cm² is an irradiance that can suppress any heat sensation or pain caused by the heat sensation that could be experienced by the patient during a therapy.

The control module 30 may be capable of controlling the driving of the LED elements 24 individually. In this case, the control module 30 can selectively turn on some of the LED elements 24 in accordance with the size and the shape of an affected area in a skin of a patient.

When the phototherapy device 10 is used, the operator holds the hand lever 19 and brings the light source module 20 to a position where the light-irradiation window 28 faces an affected area of the patient. From the viewpoint of ensuring a stable irradiance, the phototherapy device 10 is preferably used in a state where the light source module 20 (light-irradiation window 28) is in contact with or in proximity to the affected area (for example, with a space of about 3 cm). Then, the operator operates the graphic operation panel 39 of the control module 30. Thus, the LED elements 24 supplied with power from the control module 30 turn on in the light source module 20, and the affected area of the patient is irradiated (surface irradiation) with the therapeutic light.

In the phototherapy device 10, the therapeutic light emitted from the light source module 20 has a peak wavelength in the above-mentioned specific wavelength range. Therefore, as the affected area is irradiated with the therapeutic light, collagenase (MMP1), which is an enzyme that decomposes and breaks collagen causing sclerema in scleroderma, can be manifested with a significant difference. Accordingly, a superior therapeutic effect against scleroderma can be obtained.

In the phototherapy device 10, the therapeutic light emitted from the light source module 20 is light in which light having a wavelength of no longer than 350 nm is substantially cut. Therefore, even if an affected area is irradiated with this therapeutic light, immediate pigment darkening can be suppressed. This feature will be described through experimental examples below.

### Experimental Example 1

Firstly, a receptacle such as a 35-mm dish was seeded with 1×10⁵ cells of Normal Human Epidermal Melanocytes-Neonatal (HEMn), and this was cultured over twenty-four hours with the use of a constant-humidity incubator under the condition where the temperature was 37° C. and the carbon dioxide (CO₂) concentration of the atmosphere inside the incubator was 5%. Thereafter, the medium was removed, and 1 ml of saline (phosphate buffered saline (PBS)) was added.

Then, with the use of an LED light source that emits light having a peak wavelength in a wavelength range of 365 nm±5 nm, the cells were irradiated with the light to attain an irradiation amount (cumulative irradiation amount or dose) of 15 J/cm² under the irradiation condition shown in Table 1 below.

The irradiation was performed for the following four groups: (1) no irradiation (control), (2) without filter, (3) with an A filter, and (4) with a B filter.

TABLE 1

| | | Irradiation | | Irradiation |
|---|---|---|---|---|
| | Dose | Distance | Irradiance | Duration |
| Group Name | (J/cm²) | (mm) | (mW/cm²) | (h:m:s) |
| Control | 0 | — | — | 0 |
| Filter (−) | 15 | 30 | 114.4 | 0:02:15 |
| A filter (+) | 15 | 30 | 85.8 | 0:02:55 |
| B filter (+) | 15 | 30 | 97.2 | 0:02:34 |

The A filter is a filter of the example of the invention, and the B filter is a filter of a comparative example.

The filter characteristics of the A filter and the B filter are indicated by curves A and B, respectively, in FIG. 5. The characteristic values of the A filter and the B filter are shown in Table 2. The A filter has a characteristic of substantially cutting, of the incident light, light having a wavelength of around 340 nm, and the B filter has a characteristic of transmitting, of the incident light, some (around 35%) of the light having a wavelength of around 340 nm.

TABLE 2

| | A filter | B filter |
|---|---|---|
| λT (nm) | 355 | 340 |
| ΔλT (nm) | 23 | 26 |
| TH (%) | 91.0 | 90.7 |

Upon having passed through the A filter, the light having a peak wavelength in a wavelength range of 365 nm±5 nm emitted from the LED light source results in the light in which light having a wavelength of no longer than 350 nm is substantially cut. This phenomenon will be described below.

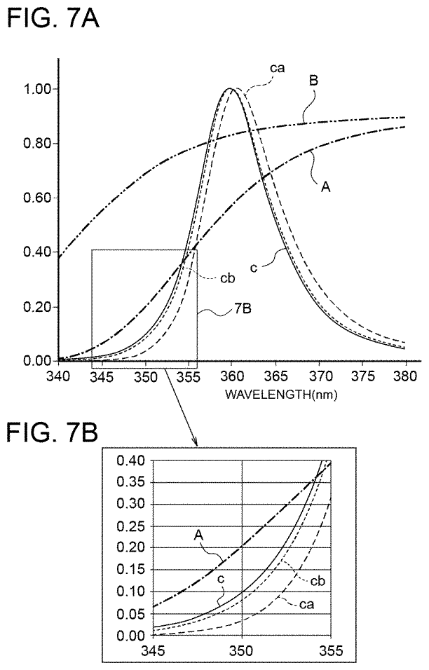

For light from an LED element at a lower limit of the variation in the center wavelength (center wavelength: 360 nm), the spectra obtained after passing through the A filter and the B filter are illustrated in FIG. 7A. FIG. 7B is a partial enlarged view of FIG. 7A. In FIGS. 7A and 7B, the solid line c represents the spectrum of the LED element having a center wavelength of 360 nm, the dashed-dotted line A represents the spectral transmittance of the A filter, the dashed-two-dotted line B represents the spectral transmittance of the B filter, the dashed line ca represents the spectrum of the light from the LED element that has passed through the A filter, and the dotted line cb represents the spectrum of the light from the LED element that has passed through the B filter. Each spectrum is normalized with the peak value being 1. The horizontal axis of the graph shown in FIG. 7A indicates the wavelength (nm).

Table 3 shows the intensity ratio of light having a wavelength of 350 nm to the peak intensity of the light from the LED element when the peak intensity of the light from the LED is regarded as 1; the intensity ratio of light having a wavelength of 350 nm to the peak intensity of the light from the LED element that has passed through the A filter when the peak intensity of the light from the LED element that has passed through the A filter is regarded as 1; and the intensity ratio of light having a wavelength of 350 nm to the peak intensity of the light from the LED element that has passed through the B filter when the peak intensity of the light from the LED element that has passed through the B filter is regarded as 1.

TABLE 3

| | Intensity Ratio of Light |
|---|---|
| | Having Wavelength of 350 |
| | nm Peak Wavelength |
| LED (Peak Wavelength: 360 nm) | 10% |
| LED × A Filter | 3% |
| LED × B Filter | 8% |

The intensity ratio of the light having a wavelength of 350 nm to the peak wavelength of the LED element is 10%, and the intensity ratio of the light having a wavelength of 350 nm to the peak wavelength of the light that has passed through the A filter is 3%. Thus, the light having a wavelength of 350 nm is substantially cut by the A filter. On the other hand, the intensity ratio of the light having a wavelength of 350 nm to the peak wavelength of the light that has passed through the B filter is 8%, and the light that has passed through the B filter includes the light having a wavelength of 350 nm.

In this manner, the A filter was provided in the LED light source, and the cells were irradiated with the light in which the light having a wavelength of no longer than 350 nm was substantially cut. In addition, the B filter was provided in the LED light source, and the cells were irradiated with the light in which the light having a wavelength of no longer than 350 nm was not cut.

Subsequently, the PBS was removed from each dish by suction, and the medium was added. This was cultured over eight days with the use of a constant-humidity incubator under the condition where the temperature was 37° C. and the CO₂ concentration of the atmosphere inside the incubator was 5%.

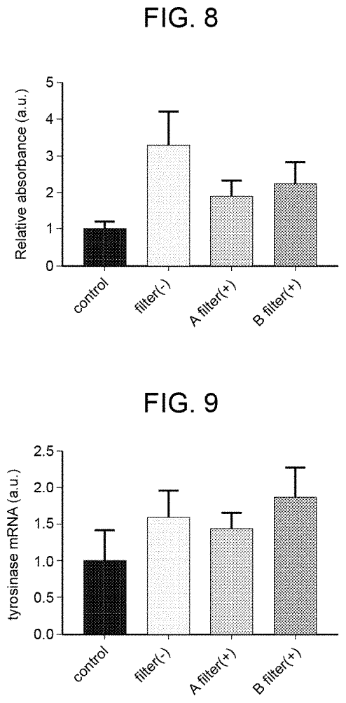

Then, the cells were collected, and the absorbance at 405 nm was measured with a plate reader (SpectraMax 340, Molecular Devices). In other words, an immediate pigment darkening reaction was observed. The result is shown in FIG. 8.

In FIG. 8, each measurement result is indicated in a relative value where the measurement result obtained in “no irradiation (control)” is regarded as 1. As can be seen from FIG. 8, the absorbance increases upon irradiating the cells with the light, that is, pigment darkening of melanin is promoted (facilitated) as the cells are irradiated with the light. In addition, it has been confirmed that this pigment darkening is promoted the most when no filter is provided and that the pigment darkening is suppressed as the filter is provided. It has also been confirmed that the pigment darkening is suppressed further when the A filter is provided than when the B filter is provided.

### Experimental Example 2

Firstly, a receptacle such as a 35-mm dish was seeded with 1×10⁵ cells of Normal Human Epidermal Melanocytes-Neonatal (HEMn), and this was cultured over twenty-four hours with the use of a constant-humidity incubator under the condition where the temperature was 37° C. and the carbon dioxide (CO₂) concentration of the atmosphere inside the incubator was 5%. Thereafter, the medium was removed, and 1 ml of saline (phosphate buffered saline (PBS)) was added.

Then, with the use of an LED light source that emits light having a peak wavelength in a wavelength range of 365 nm±5 nm, the cells were irradiated with the light to attain an irradiation amount (cumulative irradiation amount or dose) of 15 J/cm² under the irradiation condition shown in Table 1.

The irradiation was performed for the following four groups: (1) no irradiation (control), (2) without filter, (3) with an A filter, and (4) with a B filter. The characteristics of the A filter and B filter of Experimental Example 2 are the same as Experimental Example 1, i.e., the filter characteristics are shown in FIG. 5.

Subsequently, the PBS was removed from each dish by suction, and the medium was added. This was cultured over twenty-four hours with the use of a constant-humidity incubator under the condition where the temperature was 37° C. and the CO₂ concentration of the atmosphere inside the incubator was 5%.

Then, the cells were collected, and expression of mRNA of tyrosinase was measured by a real-time PCR method. In other words, an immediate pigment darkening reaction was observed. The result is shown in FIG. 9.

In FIG. 9, each measurement result is indicated in a relative value where the measurement result obtained in “no irradiation (control)” is regarded as 1. As can be seen from FIG. 9, the expression of mRNA of tyrosinase is promoted (facilitated) upon irradiating the cells with the light. In addition, it has been confirmed that the expression of mRNA of tyrosinase is more suppressed when the A filter is provided than when no filter is provided.

From the above-described Experiments, it is confirmed that when the A filter that has the filter characteristics shown in Table 2 is provided in the phototherapy device, it is possible to efficiently suppress the immediate pigment darkening action in response to the light irradiation to the affected area of the patient with the light having a peak wavelength in a wavelength range of 365 nm±5 nm. Specifically, the A filter can substantially shield the light around the wavelength of 340 nm of the light emitted from the LED light source, and consequently the side effect upon light irradiation (i.e., immediate pigment darkening action) is suppressed while obtaining a desired therapeutic effect.

As described above, the light source module 20 of the phototherapy device 10 of this embodiment includes a plurality of LED elements 24 to emit light having a peak wavelength in a wavelength range of 365 nm±5 nm, the light irradiation window (window member) 28 to which the light emitted from the LED elements 24 is incident and which allows the therapeutic light to pass therethrough, and the filter 26 located between the LED elements 24 and the light irradiation window 28 and configured to substantially shield a particular part of the light emitted from the LED elements 24 (light having a wavelength equal to or shorter than 350 nm).

In this manner, the therapeutic device 10 assumes that the peak wavelength of the light emitted in the therapeutic device 10, which uses a plurality of LED elements 24 in combination as the light source unit, may vary by around +5 nm. Based on such assumption, the therapeutic device 10 includes the filter 26 (i.e., filter to cut the short wavelength light) in order to suppress the side effect (i.e., the immediate pigment darkening action).

Therefore, the therapeutic light emitted from the light source module 20 has a peak wavelength in the predetermined wavelength range of 365 nm±5 nm while the light having a wavelength equal to or shorter than 350 nm is, in effect, being shielded. Accordingly, the therapeutic device 10 can emit light that includes light having a wavelength around 356 nm and does not include light having a wavelength around 340 nm substantially, as the therapeutic light. The light having a wavelength around 356 nm provides a great therapeutic effect to the scleroderma. The light having a wavelength around 340 nm would trigger the immediate pigment darkening action. Thus, it is possible to suppress the immediate pigment darkening action while obtaining a remarkable skin therapeutic effect to the scleroderma.

It is assumed (or predicted) that the therapy or medical treatment is repeatedly applied to the scleroderma. If the immediate pigment darkening action is repeated at every medical treatment, and the long-lasting pigmentation occurs, then the light absorption at the epidermis increases and the invasiveness (penetration) of the light into the dermis, which is the target of therapy, decreases. This reduces the therapeutic efficiency.

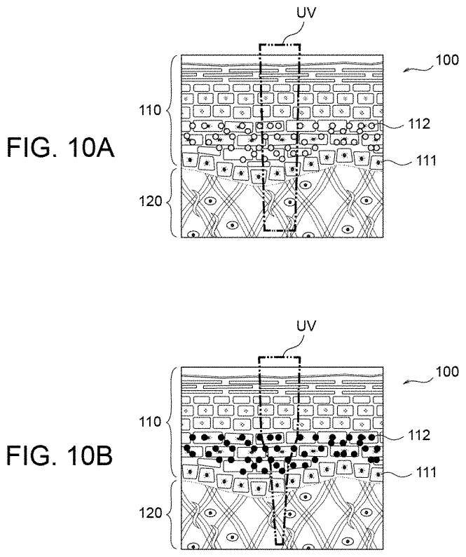

If the pigmentation does not occur, the light absorption at the epidermis 110 is small, as shown in FIG. 10A. Thus, the light (therapeutic light) UV directed to the skin 100 can reach the dermis 120, where the target cells exist. On the other hand, as shown in FIG. 10B, if the pigmentation occurs, the light is absorbed by the dark or black melanin 112, and therefore a sufficient amount of therapeutic light UV does not reach the dermis 120. As a result, the therapeutic effect is deteriorated as compared to when there is no pigmentation.

As described above, the embodiment of the invention can suppress the immediate pigment darkening action. Thus, it is possible to suppress the pigmentation, maintain the deep penetration of the light into the dermis, and maintain the therapeutic effect/efficiency.

Even if there are variations in the peak wavelength of the LED elements 24, it is possible to use such LED elements as the light source elements, without any modifications and adjustments. In other words, selection of LED elements 24 or the like is not necessary in order to prepare the LED elements that have the same peak wavelength.

If the filter 26 is a colored glass filter, the filter 26 can improve the efficiency of utilization of light, as compared to a case where the filter 26 is a dielectric multi-layer filter. Because the dielectric multi-layer filter has the incident angle dependency, it is necessary to improve the angle characteristics of the light emitted from the LED elements with a collimator lens, if the efficiency of utilization of light should be improved and the dielectric multi-layer filter should be used. This results in the complicated device configuration and the large device size. In contrast, if the colored glass filter is used as the filter 26, the device configuration becomes simple and the device size becomes small.

It should be noted that the filter 26 in the embodiment of the invention may be an arbitrary filter as long as the filter 26 can substantially shield the light at the wavelength equal to or shorter than 350 nm, preferably at the wavelength of 355 nm, of the light emitted from the LED elements 24. For example, the filter 26 may be a dielectric multi-layer filter. From the viewpoint of the efficiency of utilization of light, however, it is preferred that the colored glass filter is used as the filter 26.

The irradiance of the therapeutic light immediately below the light outlet window 28 of the phototherapy device 10 may be between 33 mW/cm², inclusive, and 150 mW/cm², inclusive. When the irradiance of the therapeutic light immediately below the light outlet window 28 is equal to or greater than 33 mW/cm², it is possible to reduce the treatment time (therapy time). When the irradiance of the therapeutic light immediately below the light outlet window 28 is equal to or smaller than 150 mW/cm², it is possible to reduce the heat and heat-derived pain, which a patient feels during the treatment.

### Modifications

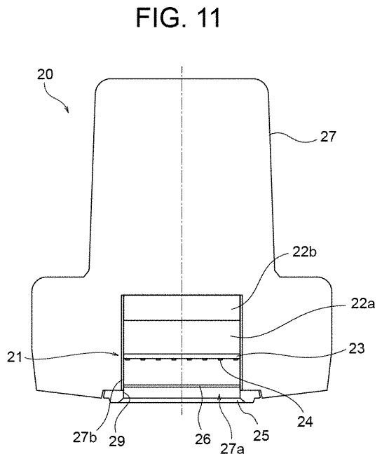

Although the filter 26 is situated between the LED elements 24 and the light outlet window 28 in the above-described embodiment, the present invention is not limited to such arrangement. For example, the filter 26 may be combined to the light outlet window 28, i.e., the filter 26 may also serve as the light outlet window 28. This modification is illustrated in FIG. 11. As shown in FIG. 11, the filter 26 is placed at the position of the light outlet window 28 of FIG. 2, and serves as the light outlet window. In this configuration, it is not necessary to provide a separate light outlet window (silica window), and therefore it is possible to reduce the cost for the light outlet window.

It should be noted that the configuration of the phototherapy device 10 is not limited to the illustrated and described configuration. It is satisfactory if the phototherapy device 10 includes the light source module 20 and the control module 30. The configuration of the light source module 20 and the configuration of the control module 30 are not limited to those illustrated in FIGS. 1 and 2. Also, the parts and elements of the phototherapy device 10 other than the light source module 20 and the control module 30 may be replaced with other parts and elements. For example, the phototherapy device 10 may have a handle, which the operator of the phototherapy device 10 can grasp to hold the phototherapy device 10 such that the operator of the phototherapy device 10 can move the light source module 20 to a desired position during the treatment, i.e., the phototherapy device 10 may be a handy-type device.

While certain embodiment and modifications have been described, these embodiments and modifications have been presented by way of example only, and are not intended to limit the scope of the present invention. The novel apparatuses and methods thereof described herein may be embodied in a variety of other forms; furthermore, various omissions, substitutions and changes in the form of the apparatuses and methods thereof described herein may be made without departing from the gist of the present invention. The accompanying claims and their equivalents are intended to cover such forms or modifications as would fall within the scope and gist of the present invention.

The present application is based upon and claims the benefit of a priority from Japanese Patent Application No. 2018-184050, filed Sep. 28, 2018, and the entire content of which is incorporated herein by reference.

What is claimed is:

1. A phototherapy device comprising:
a light source module configured to emit therapeutic light to an affected area,
the light source module including:
a plurality of LED elements to emit light having a peak wavelength in a range of 365 nm±5 nm, and having a wavelength equal to or shorter than 350 nm,
a light outlet window to which the light emitted from the plurality of LED elements is incident and through which the therapeutic light exits, and
a filter configured to substantially shield light having a wavelength equal to or shorter than 350 nm when the light emitted from the plurality of LED elements passes through the filter.

2. The phototherapy device according to claim 1, wherein the filter substantially shields light having a wavelength equal to or shorter than 355 nm when the light emitted from the plurality of LED elements passes through the filter.

3. The phototherapy device according to claim 1, wherein the plurality of LED elements have different peak wavelengths, respectively, in a range of 365 nm±5 nm.

4. The phototherapy device according to claim 1, wherein the filter is a colored glass filter.

5. The phototherapy device according to claim 4, wherein the colored glass filter has a 5% transmittance at a first wavelength and a 72% transmittance at a second wavelength, a midpoint between the first wavelength and the second wavelength is between 350 nm, inclusive, and 365 nm, inclusive, and a difference between the first wavelength and the second wavelength is equal to or shorter than 30 nm.

6. The phototherapy device according to claim 5, wherein the first wavelength is equal to or shorter than 340 nm.

7. The phototherapy device according to claim 5, wherein the colored glass filter has an average transmittance of 80% or more in a wavelength range between the second wavelength, inclusive, and 800 nm, inclusive.

8. The phototherapy device according to claim 1, wherein irradiance of the therapeutic light immediately below the light outlet window is between 33 mW/cm², inclusive, and 150 mW/cm², inclusive.

9. The phototherapy device according to claim 2, wherein the plurality of LED elements have different peak wavelengths, respectively, in a range of 365 nm±5 nm.

10. The phototherapy device according to claim 9, wherein the filter is a colored glass filter.

11. The phototherapy device according to claim 10, wherein the colored glass filter has a 5% transmittance at a first wavelength and a 72% transmittance at a second wavelength, a midpoint between the first wavelength and the second wavelength is between 350 nm, inclusive, and 365 nm, inclusive, and a difference between the first wavelength and the second wavelength is equal to or shorter than 30 nm.

12. The phototherapy device according to claim 11 wherein the first wavelength is equal to or shorter than 340 nm.

13. The phototherapy device according to claim 12, wherein the colored glass filter has an average transmittance of 80% or more in a wavelength range between the second wavelength, inclusive, and 800 nm, inclusive.

14. The phototherapy device according to claim 13, wherein irradiance of the therapeutic light immediately below the light outlet window is between 33 mW/cm², inclusive, and 150 mW/cm², inclusive.

15. A method of emitting therapeutic light to an affected area from a light source module having a plurality of LED elements, the method comprising:
causing the plurality of LED elements of the light source module to emit light having a peak wavelength in a range of 365 nm±5 nm, and having a wavelength equal to or shorter than 350 nm;
substantially shielding, by a filter, light having a wavelength equal to or shorter than 350 nm when the light emitted from the plurality of LED elements passes through the filter; and
directing the light, which has been emitted from the plurality of LED elements and has passed through the filter, to the affected area as the therapeutic light.
